(19) United States
(12) Patent Application Publication
(10) Pub. No.: US 2023/0338620 A1
(43) Pub. Date: Oct. 26, 2023

(54) INJECTABLE COMPOSITION FOR BONE REGENERATION

(71) Applicant: Denhouse Co., Ltd, Jeollabuk-do (KR)

(72) Inventor: Kyung-hwan Kwon, Jeollabuk-do (KR)

(73) Assignee: Denhouse Co., Ltd, Jeollabuk-do (KR)

(21) Appl. No.: 18/003,073

(22) PCT Filed: Jun. 25, 2021
(86) PCT No.: PCT/KR2021/008010

§ 371 Date: Dec. 22, 2022

(30) Foreign Application Priority Data
Jun. 26, 2020 (KR) 10-2020-0078166

(51) Int. Cl. A61L 27/54; A61L 27/24

(52) U.S. Cl. CPC A61L 27/54; A61L 27/24; A61L 2430/02; A61L 2400/06; A61L 2300/254; A61L 2300/402

(57) ABSTRACT

The present invention relates to an injectable composition for bone regeneration, comprising 1 cc of 6% (w/v) hydrated collagen, 5000 units of thrombin, 0.5 ml of a mixed solution of lidocaine and epinephrine in which the epinephrine amount is 1:100,000 (v/v) on the basis of lidocaine, and acting by being injected into bone marrow. The composition according to the present invention activates undifferentiated cells in bone marrow and also activates the bone regeneration ability in bone marrow through the activation and differentiation of osteoclasts and osteoblasts, and can be a successful treatment method for medication-related osteonecrosis of the jaws (MRONJ) through a myeloid activation treatment method, uses various growth factors for myeloid activation and myeloid activation factors such as cytokines so as to allow for the development of treatment for the fundamental cause of MRONJ through a myeloid activation treatment method, and enables the complete regeneration of jaw bone conditions with respect to aesthetic and function, and thus can be effectively used in the treatment of various diseases caused by bone remodeling failure.

### TECHNICAL FIELD

The present invention, which relates to an injectable composition for bone regeneration, relates to an injectable bone regeneration composition capable of activating undifferentiated cells in bone marrow and also activating bone regeneration capacity in bone marrow through the activation and differentiation of osteoclasts and osteoblasts.

### BACKGROUND ART

In the dental field, innovative treatment methods have constantly developed as clinical knowledge on bone has constantly developed and tissue engineering in the bone regeneration field along with molecular biology has developed. However, recently, therapeutic agents for osteoporosis, anti-angiogenic drugs, steroid immunosuppressive agents, therapeutic agents for hyperlipidemia, etc., are widely used for long term, which leads to various intractable diseases such as osteonecrosis or osteomyelitis. Thus, it is not easy to conduct treatment in practice.

The first discovery of osteonecrosis caused by alendronate injections in 2003 by Marx (Marx R E (September 2003). “Pamidronate (Aredia) and zoledronate (Zometa) induced avascular necrosis of the jaws: a growing epidemic” J. Oral Maxillofac. Surg. 61 (9): 1115-7), and bisphosphonate (the drug for treatment of osteoporosis)—related osteonecrosis was also discovered in Korea after 2005. It was misidentified as a type of osteomyelitis and treated by a method for treating osteomyelitis. However, osteomyelitis or osteonecrosis symptoms persisted, and side effects of invading into larger area and complications occurred.

Case Presentation 1-1 of a patient with multiple myeloma misidentified as osteomyelitis in 2005, who was administered with an injection of Actonel (a bisphosphonate): Osteonecrosis in the jaw or local necrosis of bone tissue were rare potential complications for patients with cancer who received radiotherapy or chemotherapy, or received treatment for patients with tumor or infectious embolism. In 2003, there was a report that a risk of osteonecrosis increased in patients who were treated in combination with bisphosphonate intravenous administration.

Matrix metalloproteinase 2 may be a candidate gene for bisphosphonate-related osteonecrosis in the jaw, and it is the only gene involved in bone abnormalities and atrial fibrillation. Thus, there is a report that it may cause another side effect of bisphosphonates.

In 2005, the US Food and Drug Administration (FDA) released a broader drug class warning of osteonecrosis complications for all bisphosphonates.

Since 2007, through multi-instrumental examination by Marx on patients who took a broad range of bisphosphonates, it was reported that extensive osteonecrosis was being occurred. Also, there have been reports in Korea since 2007 announcing that osteonecrosis has occurred. However, the studies on principle or mechanism of medication-related osteonecrosis were not sufficient. In particular, bone biology has developed mostly regarding the studies on osteoblasts.

The recently revealed cause of osteonecrosis was recognized as phenomenon associated with malfunction of osteoclasts, thereby resulting in hindrance of the chemical migration or differentiation of osteoblasts with bone regeneration capacity. As such, it was known that the activity of osteoclasts, rather than an increase in the number of osteoblasts, plays a crucial role in bone regeneration.

Bone cells include osteoblasts, osteocytes, and osteoclasts. In the fields of physiology or biology studies on bone since 2000, attention has focused on an increase in the number of osteoblasts and osteocytes using stem cells or undifferentiated cells and an increase in the production rate of bone.

In particular, as for bone grafting for dental implants, attention has focused on bone materials that can increase osteocytes. However, the main attention regarding these bone materials has focused on osteoconduction. As for the treatment of bone for osteoporosis or rheumatoid arthritis, the studies were focused on inhibition of the number or action of osteoclasts, and it was vaguely agreed that osteoporosis or rheumatoid arthritis could be treated by such treatment methods.

Since 2010, both researchers who study bone biology, bone grafting, or bone curing and clinicians have proved that increasing or decreasing the number of osteocytes or osteoclasts is not the way of improving the success rate of bone curing or bone grafting.

With the facts alone that side effects of bisphosphonates widely used to treat osteoporosis broadly increase, and that various drugs, steroids, and nonopioid analgesics used to treat rheumatoid arthritis cause side effects of inducing osteonecrosis and reducing bone immunity, a need for development of novel studies are being spread.

Bone is a composite tissue of proteins and minerals and is continuously remodeling itself. Osteoclasts, bone absorbing materials and osteoblasts which form bone play a role in bone growth, damage treatment, regulation in the metabolism of calcium and phosphate and absorb the bone during remodeling. They are recognized as basic multicellular units for forming the bone.

Osteoclasts are large multinucleated cells distinguished from bone marrow precursors, controlled by macrophage colony-stimulating factor (MCSF) (cytokines provided by osteocytes) and receptor activator of NF NF-KB ligand (RANKL). Osteoclasts hydrolyze proteinase (e.g., cathepsin K) and acid, and digest and dissolve organic and inorganic components of the bone matrix.

When the coupling between osteoclasts and osteoblasts is broken, the delicate balance between the bone resorption activities and the bone formation activities is lost resulting in bone diseases. Such phenomena are reported as side effects of bisphosphonates when used as the therapeutic agent for patients with osteoporosis. Actonel, Bonviva, etc., which are bisphosphonate drugs initially based on the mechanism of treating osteoporosis by reducing reabsorption mechanism to induce bone loss, destroyed the coupling network relationship between osteoclasts and osteoblasts and disabled the mechanism for bone regeneration.

Osteonecrosis of the jaw (ONJ) is an oral disease accompanying the exposure of the jaw bone. Some patients may experience no symptoms, but ONJ generally involves pain and pus discharge.

This symptom may occur naturally, or after extraction of a tooth, wounds, or radiotherapy to the head and neck (referred to as osteoradionecrosis).

ONJ may be actually bone infection (osteomyelitis), not osteonecrosis. ONJ is observed in people to whom recently a high dose of bisphosphonates is administered intravenously, in particular people who suffered from cancer or received oral surgery while being administered with this drug.

There were reports that ONJ is not associated with the ordinary use of bisphosphonates orally administered for treatment of osteoporosis. However, recently it was informed that in the case of long-term use of the drug for 3 years or more, osteonecrosis occurs. It was revealed that such symptoms are caused by malfunction of osteoclasts.

In Korea, it has been reported that the prevalence rate of bisphosphonate-related osteonecrosis of the jaw (BRONJ), the representative disease of medication-related osteonecrosis of the jaw, is about between 2 and 10% in patients who were orally administered with bisphosphonates for 3 years or more or with an injection thereof for 1 year or more. It is known that the administration of drugs in combination amplifies the rate.

In particular, it was reported that when administering steroids used to treat arthritis and bisphosphonates in combination or when simultaneously administering the therapeutic agents for hyperlipidemia and bisphosphonates, the prevalence rate of osteonecrosis further increased. Thus, it is most important for dental clinic to analyze whether drugs are administered and to assay the components thereof.

Bones are always regenerated by osteoclasia and osteogenesis. Bones providing support for the human body act as reserves of minerals such as calcium. When renewing bones, old bones should be broken down by osteoclasts.

When osteoclasts do not function properly, interleukins such as IL-1 and IL-6 are not secreted, and the cells do not play a role in the differentiation and regeneration of osteoblasts of interleukins, failing to bone regeneration. Further, osteoblasts strongly express receptor activator of nuclear factor kappa-B ligand (RANKL) necessary for raising osteoclasts. It could be confirmed from experiments that in mice having osteoblasts with RANKL removed, osteoclasts do not grow and severe marble bone diseases occur.

### SUMMARY OF THE INVENTION

### Technical Objective

Therefore, the objective that the present invention intends to solve is to provide an injectable bone regeneration composition capable of activating undifferentiated cells in bone marrow and also activating bone regeneration capacity in bone marrow through the activation and differentiation of osteoclasts and osteoblasts.

### Means for Solving Technical Objective

In order to solve the above objective, the present invention provides an injectable composition for bone regeneration acting by being injected into the bone marrow, the composition comprising 1 cc of 6% (w/v) hydrated collagen; 5000 units of thrombin; and 0.5 ml of a mixed solution of lidocaine and epinephrine in which the content of epinephrine is 1:100,000 (v/v) relative to lidocaine.

### Working Effect of the Invention

The composition according to the present invention, which activates undifferentiated cells in bone marrow and also activates bone regeneration capacity in bone marrow through the activation and differentiation of osteoclasts and osteoblasts, may be a method for successful treatment of medication-related osteonecrosis of the jaw (MRONJ) through a bone marrow activation treatment method, and may develop the treatment of the fundamental causes of MRONJ through a bone marrow activation treatment method using various growth factors for bone marrow activation and bone marrow activation factors such as cytokines and completely regenerate jaw bone conditions aesthetically and functionally. Accordingly, the composition according to the present invention may be useful in the treatment of various diseases caused by bone remodeling failure.

### BRIEF DESCRIPTION OF DRAWINGS

FIG. 1: a picture of a patient complaining pain, having a fistula in the skin on the mandible discharging pus.

FIG. 2: a panoramic picture indicating that the radiographic image and the radiopaque image, which show the osteonecrosis of bone marrow in the right mandible, are present in the boundary of osteonecrosis.

FIG. 3: a picture showing that pus is discharging into the oral cavity.

FIG. 4: a picture showing removing the alveolar crest and approaching the bone marrow to remove the necrotic bone marrow.

FIG. 5: a picture filling a therapeutic component for bone marrow activation (OSSCORE LMT collagen scaffold) into the bone marrow while allowing the blood to be smoothly supplied into the bone marrow through cortical perforations in the buccal cortical bone and the lingual cortical bone.

FIG. 6: a picture showing a suture made such that the bone marrow site and the bone marrow activation scaffold are not exposed using a continuous locking suture and an interrupted suture in combination.

FIG. 7a: a facial picture 6 months after surgery, showing that the skin fistula in the right mandible disappears.

FIG. 7b: a picture showing that, according to the doctor's opinion, no pus discharge is present in the oral cavity and that the primary suture has been successfully made at the suture site 6 months after surgery.

FIG. 7c: a cone beam 3D CT image before surgery and a cone beam 3D CT image 6 months after surgery.

FIGS. 8a, 8b and 8c: pictures showing the process of treating patients of other cases using the composition according to the present invention.

### DETAILED DESCRIPTION OF THE INVENTION

Hereinafter, the present invention will be described in detail.

The present invention provides an injectable composition for bone regeneration, comprising hydrated collagen; and a mixed solution of lidocaine, epinephrine and thrombin.

The thrombin promotes proliferation of osteoblasts while inhibiting apoptosis thereof via activation of protease-activated receptor-1 (PAR-1). The promotion serves as osteoprotegerin (OPG) which inhibits collagen secretion and RANKL using osteoblasts. Thrombin plays a role at the initial stage of bone regeneration, allowing the differentiation of osteoblasts and the inhibition of osteoclasts at the initial stage. When lidocaine-thrombin-collagen, which is a bone marrow activation scaffold, is adequately mixed, pain or inflammation induction at the initial stage is reduced and vascularization is induced, and the vascularization positively stimulates the migration of undifferentiated stem cells.

Lidocaine is an amide-type local anesthetic synthesized by Swedish chemists Nils

Lofgren and Bengt Lundqvist in 1943. Lidocaine is slightly inferior in terms of action or duration to tetracaine known as the strongest local anesthetic. However, lidocaine is less toxic than tetracaine and has sufficient efficacy. Thus, lidocaine is a local anesthetic generally safe for use in local anesthesia. It may be used with the addition of epinephrine to prolong its effects as an anesthetic in a small amount and to suppress bleeding by hemostatic action during surgery, by contracting blood vessels around the anesthetized site. It has high fat-soluble protein binding capacity and prolonged duration, which allows good dissolution with rhBMP-2 and collagen proteins, and thus it may also be used as a solvent.

It may induce the formation into a sustained release capsule through ionic bonding in constituting a sustained-release type scaffold along with the effect of inhibiting initial inflammatory response caused by thrombin. Using lidocaine as a solvent provides various advantages in clinical uses.

First, it has the effect of significantly reducing the occurrence of pain caused by inflammatory conditions. Second, it has the effect of slowly releasing the drug which is dissolved in a lidocaine solvent.

In addition, as epinephrine-containing lidocaine is used, it may reduce the amount absorbed into the neighboring tissues during the maximum length of time of one and a half hours in which the drug is released.

Third, it neutralizes acidic collagen solution, which leads to a change into a hard lump in which the solidity of crystals overall increases. With these three roles and advantages, the function of a scaffold of the LT collagen can be provided.

When a mixture of thrombin and lidocaine is implanted with the components of the bone marrow activation scaffold of the present invention within the initial 2 hours, hemorrhage in bone marrow is decreased and minimum edema is exhibited, which indicate the bone curing.

As a need for the sustainable development of bone graft using thrombin has been fulfilled, an injectable bone regeneration composition utilizing the initial process for bony tissue and various bone regeneration capacities of thrombin is useful.

The injectable composition for bone regeneration according to the present invention as described above is characterized in that the collagen content of the hydrated collagen is 3 to 6% (w/v).

The injectable composition for bone regeneration according to the present invention as described above is characterized in that the content of lidocaine in the mixed solution is 1 to 3% (w/v).

The injectable composition for bone regeneration according to the present invention as described above is characterized in that the content of epinephrine in the mixed solution is 1:50,000 to 200,000 (v/v) relative to lidocaine.

The injectable composition for bone regeneration according to claim 1 of the present invention as described above is characterized in that the content of thrombin in the mixed solution is 1 to 3% (w/v).

The injectable composition for bone regeneration according to the present invention as described above is characterized in that the ratio of the hydrated collagen and a mixed solution of the lidocaine, epinephrine and thrombin is 1:0.3 to 1.7 (w/w).

The above-described content ratios are determined based on various experiments and experience of the inventors of the present invention, and are the optimal ratios for achieving the best effect of the present invention.

The composition according to the present invention, which activates undifferentiated cells in bone marrow and also activates bone regeneration capacity in bone marrow through the activation and differentiation of osteoclasts and osteoblasts, may be a method for successful treatment of medication-related osteonecrosis of the jaw (MRONJ) through a bone marrow activation treatment method, and may develop the treatment of the fundamental causes of MRONJ through a bone marrow activation treatment method using various growth factors for bone marrow activation and bone marrow activation factors such as cytokines and completely regenerate jaw bone conditions aesthetically and functionally. This will be described in detail with the following examples.

### Case Patients and Treatment Methods

### Case Presentation 1

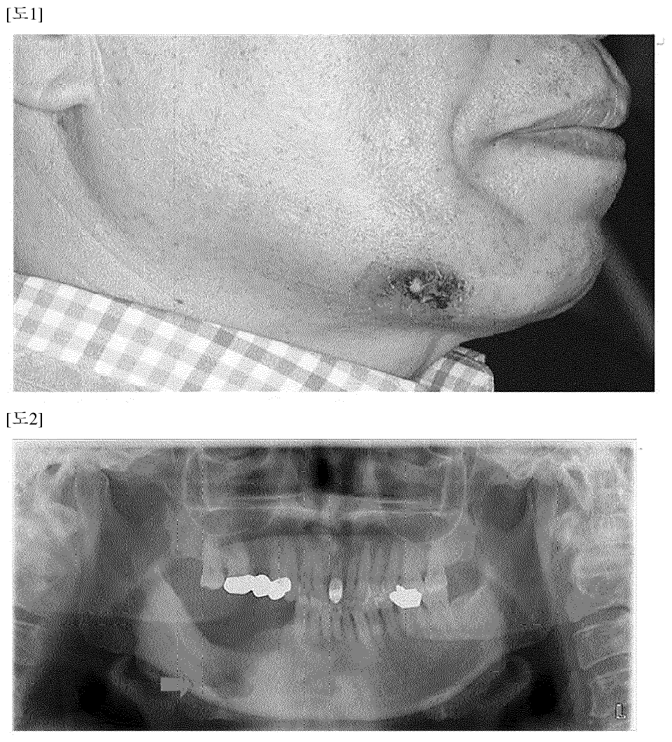

A 56-year-old man with multiple myeloma was treated with Actonel, which is a bisphosphonate, as an injection for 3 years to treat cancer and prevent jawbone metastasis. The patient had pain in the right mandible and symptoms of pus discharge along with a fistula in the skin while yellow pus discharged into the oral cavity from a year ago. Thus, the relevant oral and maxillofacial surgery department in the hospital did continuous disinfection and drainage. However, pus discharged continuously and the patient complained pain (see FIG. 1).

Drainage was performed along with removal of dead bone tissue under five local anesthesias, and treatment was conducted through primary suture. However, skin fistula and pus discharge into the oral cavity continued. According to the classification guidelines of medication-related osteonecrosis of the jaw by the American Association of Oral and Maxillofacial Surgeons, the patient is diagnosed as Stage III. It could be confirmed from the panorama and cone beam CT images that osteonecrosis and osteomyelitis pus remained in the bone marrow (see FIG. 2).

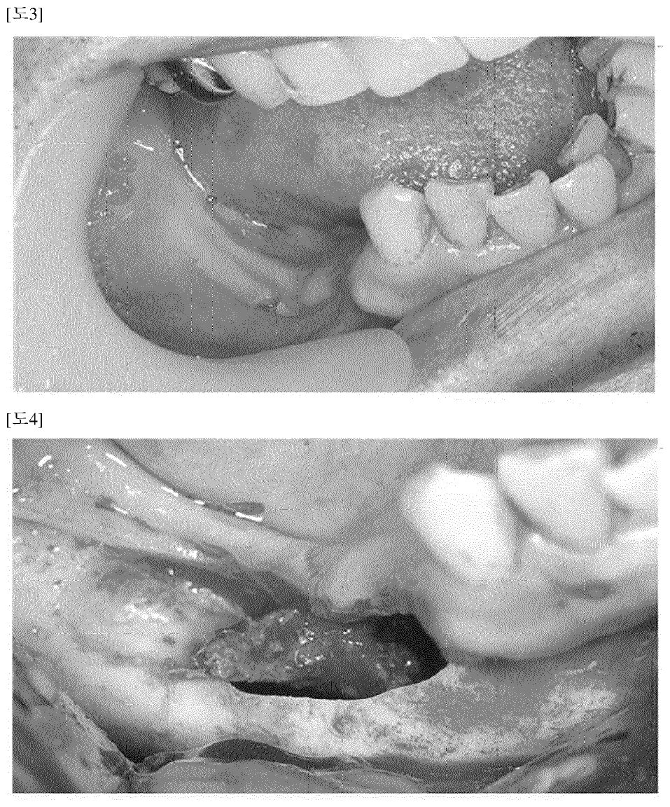

Also, FIG. 3 shows that pus discharges into the oral cavity.

### Treatment Method Using Composition of the Present Invention

An incision was made on the alveolar crest. A necrotic bone is removed using a round-shaped bur, while letting the buccal cortical bone and the lingual cortical bone remain to the maximum and removing the alveolar crest and approaching the bone marrow, thereby performing removal to the deepest site of the bone marrow as possible. The dead bone tissue in the bone marrow is removed after letting the buccal cortical bone, the lingual cortical bone, and the cortical bone present in the base wall of the mandible or maxilla remain. The cortical perforation is performed such that at least 4 to 5 perforations are made in each of the buccal cortical bone and the lingual cortical bone for each tooth position, allowing the blood to rise to the bone marrow (see FIG. 4).

In a way of maintaining the buccal cortical bone and the lingual cortical bone, and removing the alveolar crest site and approaching the bone marrow, the bone marrow tissue is removed while approaching the inferior alveolar nerve by completely removing the necrotic bone marrow tissue and protecting the inferior alveolar nerve.

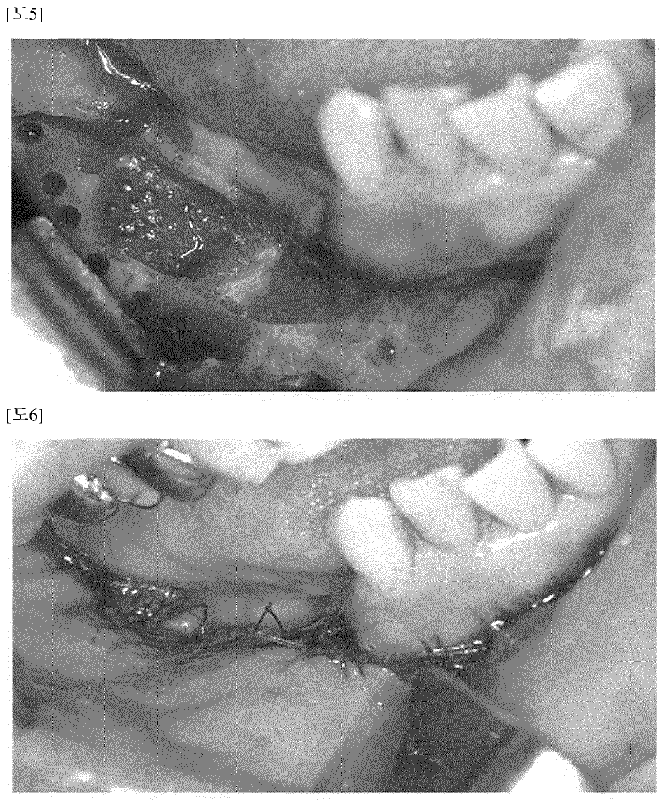

The composition of the therapeutic agent is as follows: 1.5 ml of a collagen-thrombin-lidocaine complex mixture (OSSCORE LMT collagen scaffold, Lidocaine-Maltodextrin-Thrombin collagen) is made by mixing thrombin (thrombin lyophilized powder 5000 unit—Reyon Pharmaceutical Co., Ltd., pharmaceutical product extracted from bovine plasma, imported from Germany), lidocaine (using 0.5 ml of 1.8 ml containing epinephrine 1:100,000), and 1 cc of 6% hydrated collagen (OSSCORE Denhouse). The mixture is filled by a syringe in the defect site where the bone marrow was removed, and a lidocaine-thrombin (LT) solution is sprayed onto the plug-shaped collagen to fill the whole large volume of the bone marrow (see FIG. 5).

In other words, through the cortical perforations made in the buccal cortical bone and the lingual cortical bone, a bone marrow activation treatment component (OSSCORE LMT collagen scaffold) was filled in the bone marrow, while allowing the blood to be smoothly supplied to the bone marrow. The absorbent collagen membrane soaked with the lidocaine-thrombin solution was lined around the periosteum on the cortical bone, thereby protecting the bone marrow activation component.

After filing the bone marrow activation scaffold, the incision site was sutured using a continuous locking suture and an interrupted suture in combination, so that the bone marrow site is not exposed (see FIG. 6). The suture was made using Vicryl 4-0 absorbable suture material and maintained for at least 2 weeks.

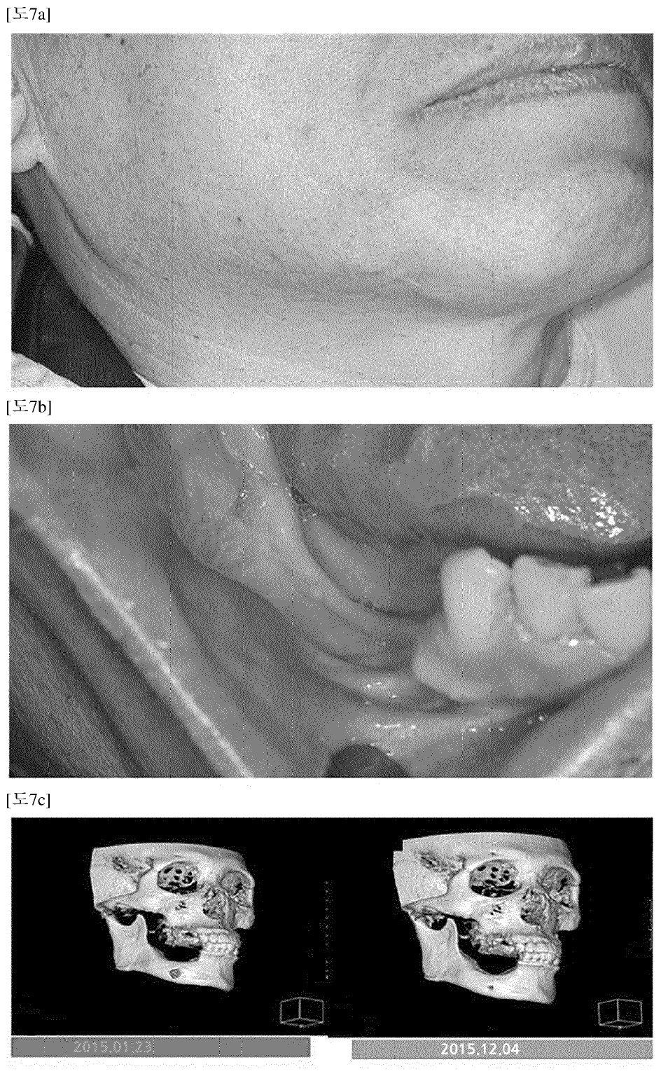

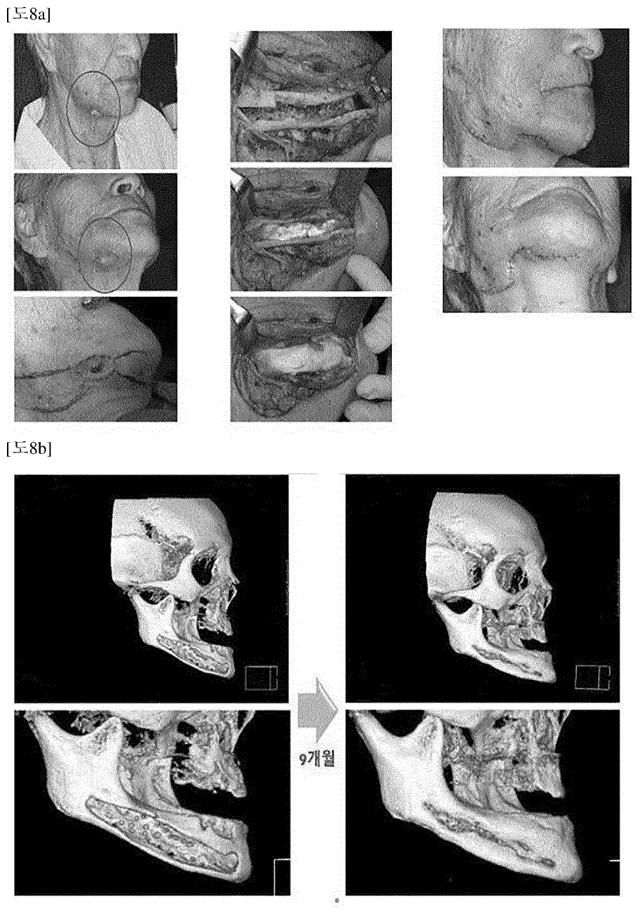

The patient was administered with penicillin antibiotics and clindamycin antibiotics in combination, and the administration was retained for 7 days after surgery. The suture material of the loose portion was removed after 2 weeks or more, and the patient was allowed to take soft foods until the mucous membrane was completely sutured and not to take out his dentures. The patient was examined persistently at 1 month, 3 months, 6 months and 12 months, and the disease has not recurred for 5 years (see FIGS. 7-a, b, c).

FIG. 7a is a facial picture 6 months after surgery, showing that the skin fistula in the right mandible disappears. FIG. 7b is a picture from which it can be observed that, according to the doctor's opinion, there is no pus discharge into the oral cavity and that the primary suture has been successfully made at the suture site 6 months after surgery.

Further, FIG. 7c is a cone beam 3D CT image before surgery and a cone beam 3D CT image 6 months after surgery. The success of the surgical procedure was confirmed from the clinical observation and radiological observation. It could be confirmed that the necrotic tissue of the bone marrow was removed, and that the bone marrow and the cortical bone have been perfectly regenerated through the bone marrow activation treatment method.

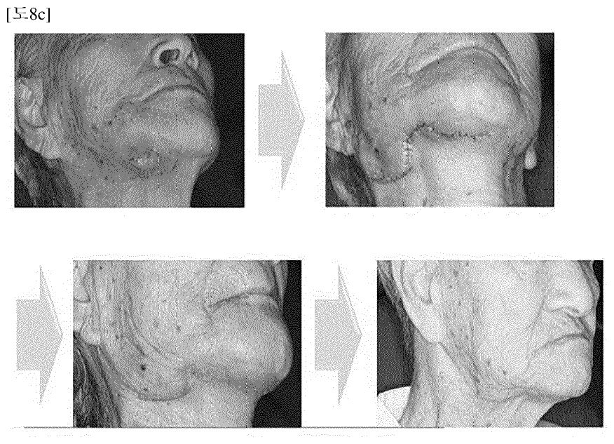

The drawings for other patients who underwent similar treatment processes are provided in FIGS. 8a, 8b and 8c. From these examples, the effect of the composition according to the present invention could also be confirmed.

What is claimed is:

1. An injectable composition for bone regeneration acting by being injected into the bone marrow, the composition comprising:
1 cc of 6% (w/v) hydrated collagen;
5000 units of thrombin; and
0.5 ml of a mixed solution of lidocaine and epinephrine in which the content of epinephrine is 1:100,000 (v/v) relative to lidocaine.
